(19) United States
(12) Patent Application Publication
(10) Pub. No.: US 2024/0050130 A1
(43) Pub. Date: Feb. 15, 2024

(54) CONSTANT DISTRACTION FORCE DRIVEN SELF ACTUATING GROWING ROD SYSTEMS

(71) Applicant: Indius Medical Technologies Private Limited, Pune (IN)

(72) Inventors: Aditya Ingalhalikar, Pune (IN); Sagar Sathaye, Pune (IN); Manali Kunte, Pune (IN); Piyali Gokhale, Pune (IN); Dhrumil Shekhda, Surat (IN)

(73) Assignee: Indius Medical Technologies Private Limited, Pune, Maharashtra (IN)

(21) Appl. No.: 18/230,473

(22) Filed: Aug. 4, 2023

(30) Foreign Application Priority Data
Aug. 10, 2022 (IN) 202221045675

(51) Int. Cl. A61B 17/70

(52) U.S. Cl. CPC A61B 17/7014; A61B 2017/00876

(57) ABSTRACT

The present disclosure relates to a constant distraction force driven self-actuating growing rod system (100) for implantation on a deformed bony anatomy. The system (100) comprises at least one static rod component (10); at least one intermediate rod component (20); at least one growth rod component (30); at least one compression spring component (40) adapted to apply an active distraction force onto the system (100) of the present disclosure; at least one magnetic field-based spring actuation mechanism (50) and at least one dynamic sealing plug (60). During the natural growth of the deformed bony anatomy, the growth rod component (10) telescopes out of the intermediate rod component (20), creating a distraction force deficit in the system (100); the distraction force deficit being corrected by causing the compression spring component (40) to get compressed by means of the magnetic field-based spring actuation mechanism (50); thereby maintaining an active distraction force onto the system (100).

### CROSS-REFERENCE TO RELATED APPLICATION

The present application takes priority from the previously filed Indian Provisional Application number 202221045675 titled “CONSTANT DISTRACTION FORCE DRIVEN SELF ACTUATING GROWING ROD SYSTEMS” dated Aug. 10, 2022.

### FIELD

The present disclosure relates to medical implants. More particularly, the present disclosure relates to constant distraction force driven self-actuating growing rod systems, to be used as medical implants.

### BACKGROUND

A common form of treatment for skeletal deformities is manual correction of the deformity by a surgeon, followed by affixation of medical implants onto the now-corrected bony anatomy, which continues to support the corrected bony anatomy. However, most of the currently existing implant systems support the corrected bony anatomy only till the point up to which natural growth occurs. Every time the patient undergoes natural growth, for instance, elongation of the spine, a surgeon has to cut open the patient and perform manual distraction of the medical implant to match the natural growth. Therefore, these systems are not natural growth driven and are highly invasive. It is crucial to note that it is the surgeon/practitioner who decides how much the system is to be distracted post implantation, and therefore, instances of human error are also inevitable.

There are certain systems that are natural growth-friendly, without the need for surgical intervention. However, such systems are fluid-based and require extra measures such as seals to mitigate the chance of fluid leakage, which increases the overall weight and cost of the system. Furthermore, if the fluid leaks, the distraction force is lost. Furthermore, growth friendly fluid-based designs of the prior are necessarily accompanied by minimally invasive procedures to increase the distraction force provided by the system as the patient grows.

The inventors of the present disclosure have envisaged a constant distraction force driven self-actuating growing rod system which mitigates the drawbacks of existing systems of the prior art.

### Objects

It is an object of the present disclosure to provide a constant distraction force driven self-actuating growing rod system.

It is another object of the present disclosure to provide a constant distraction force driven self-actuating growing rod system for orthopedic deformity correction.

It is yet another object of the present disclosure to provide a constant distraction force driven self-actuating growing rod system which is safe.

It is still another object of the present disclosure to provide a constant distraction force driven self-actuating growing rod system which is leak-proof.

It is yet another object of the present disclosure to provide a constant distraction force driven self-actuating growing rod system which is economical.

It is still another object of the present disclosure to provide a constant distraction force driven self-actuating growing rod system which mitigates or eliminates the need for repetitive surgical interventions.

### SUMMARY

The present disclosure provides a constant distraction force driven self-actuating growing rod system for implantation on a deformed bony anatomy; said system comprises at least one static rod component; at least one intermediate rod component; at least one growth rod component; at least one compression spring component adapted to apply an active distraction force onto the system of the present disclosure; at least one magnetic field-based spring actuation mechanism; and at least one dynamic sealing plug. During the natural growth of the deformed bony anatomy, the growth rod component telescopes out of the intermediate rod component, causing the pre-compressed compression spring(s) to relax; creating a distraction force deficit in the system; said distraction force deficit being corrected by causing the relaxed compression spring(s) to regain compression by means of said magnetic field-based spring actuation mechanism; thereby maintaining an active distraction force onto the system.

### BRIEF DESCRIPTION OF THE ACCOMPANYING DRAWINGS

The present disclosure is illustrated in the accompanying non-limiting drawings, throughout which reference letters indicate corresponding parts in the various figures.

FIG. 1 illustrates a non-limiting embodiment of the constant distraction force driven self-actuating growing rod system (100) of the present disclosure.

FIG. 2 illustrates a first embodiment of the constant distraction force driven self-actuating growing rod system (100) of the present disclosure at installation, wherein the growth rod component (30) is at zero distraction and the compression spring component (40) is in a pre-compressed state.

FIG. 3 illustrates the first embodiment of the constant distraction force driven self-actuating growing rod system (100) of the present disclosure pursuant to the natural growth of the deformed bony anatomy, wherein the growth rod component (30) is extended and the compression spring component (40) is compressed.

FIG. 4 illustrates a second embodiment of the constant distraction force driven self-actuating growing rod system (100) of the present disclosure at installation, wherein the growth rod component (30) is at zero distraction, the first compression spring (41) is in a pre-compressed state and the second compression spring (42) is in a free state.

FIG. 5 illustrates a second embodiment of the constant distraction force driven self-actuating growing rod system (100) of the present disclosure pursuant to the natural growth of the deformed bony anatomy, wherein the growth rod component (30) is extended, and the first compression spring (41) is compressed and the second compression spring (42) is also in a compressed state.

FIG. 6 illustrates a third embodiment of the constant distraction force driven self-actuating growing rod system (100) of the present disclosure at installation, wherein the growth rod component is at zero distraction and the compression spring component (40) is in a pre-compressed state.

FIG. 7 illustrates the third embodiment of the constant distraction force driven self-actuating growing rod system (100) of the present disclosure pursuant to the natural growth of the deformed bony anatomy, wherein the growth rod component (30) is extended, and the compression spring component (40) is compressed.

FIG. 8 illustrates a fourth embodiment of the constant distraction force driven self-actuating growing rod system (100) of the present disclosure at installation, wherein the growth rod component (30) is at zero distraction, the first compression spring (41) is in a pre-compressed state and the second compression spring (42) is in a free state.

FIG. 9 illustrates a fourth embodiment of the constant distraction force driven self-actuating growing rod system (100) of the present disclosure pursuant to the natural growth of the deformed bony anatomy, wherein the growth rod component (30) is extended, and the first compression spring (41) is compressed, and the second compression spring (42) is also in a compressed state.

FIG. 10 illustrates a fifth embodiment of the constant distraction force driven self-actuating growing rod system (100) of the present disclosure at installation, wherein the growth rod is at zero distraction and the compression spring component (40) is in a pre-compressed state.

FIG. 11 illustrates a fifth embodiment of the constant distraction force driven self-actuating growing rod system (100) of the present disclosure pursuant to the natural growth of the deformed bony anatomy, wherein the growth rod component (30) is extended, and the compression spring component (40) is compressed.

FIG. 12 illustrates a sixth embodiment of the constant distraction force driven self-actuating growing rod system (100) of the present disclosure at installation, wherein the growth rod component (30) is at zero distraction, the first compression spring (41) is in a pre-compressed state and the second compression spring (42) is in a free state.

FIG. 13 illustrates a sixth embodiment of the constant distraction force driven self-actuating growing rod system (100) of the present disclosure pursuant to the natural growth of the deformed bony anatomy, wherein the growth rod component (30) is extended, and the first compression spring (41) is compressed, and the second compression spring (42) is also in a compressed state.

FIG. 14 illustrates a cross sectional view of the fifth and/or sixth embodiment of the constant distraction force driven self-actuating growing rod system (100) of the present disclosure.

### DESCRIPTION

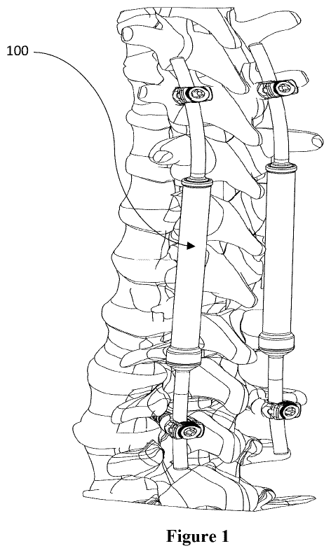

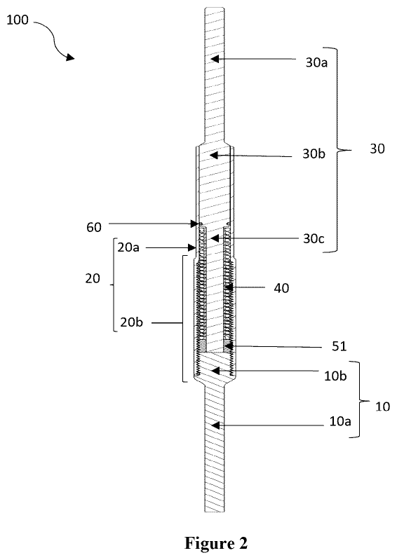

The present disclosure provides a constant distraction force driven self-actuating growing rod system (100) for implantation on a deformed bony anatomy, as illustrated in FIG. 1. FIGS. 2-14 illustrate different embodiments of the system (100) with its sub-components and their characteristic construction.

The system (100) of the present disclosure comprises at least one static rod component (10); at least one intermediate rod component (20); at least one growth rod component (30); at least one compression spring component (40); at least one magnetic field-based spring actuation mechanism (50) and at least one dynamic sealing plug (60) as the major components. The present system (100) is designed for the correction of orthopaedic deformities. In one embodiment, the present system (100) is designed for the correction of deformities in the spine.

The basic functioning of the present system (100) is described herein below. The growth rod component (30) and the static rod component (10) are adapted to be affixed to the deformed bony anatomy at pre-determined locations. The intermediate rod component (20) is hollow and is coupled threadedly with the static rod component (10) and is adapted to house the compression spring component (40) and at least one part of the magnetic field-based spring actuation mechanism (50) and optionally, the growth rod component (30). The compression spring component (40) is installed in a pre-compressed state. For the purpose of the present disclosure, the sentence that the ‘compression spring component (40) is installed in a pre-compressed state’ is to be interpreted to mean that the compression spring is installed in a way that it always exerts a certain force and is never in a free state. Additionally, when the compression spring component (40) is installed in a pre-compressed state, the total length of the compression spring component is less than the free length. The compression spring component (40) is adapted to apply an active distraction force onto the system (100) of the present disclosure. During the natural growth of the deformed bony anatomy, the growth rod component (30) telescopes out of the intermediate rod component (20), causing the pre-compressed part of the compression spring component (40) to relax; thereby creating a distraction force deficit in the system (100). The distraction force deficit is corrected by causing at least one part of the compression spring component (40) to regain compression by means of the magnetic field-based spring actuation mechanism (50); thereby maintaining an active distraction force onto the system (100). The present system (100) further comprises at least one anti-rotation feature selected from the group consisting of gears, splines, keys, ratchet, or any other suitable mechanism.

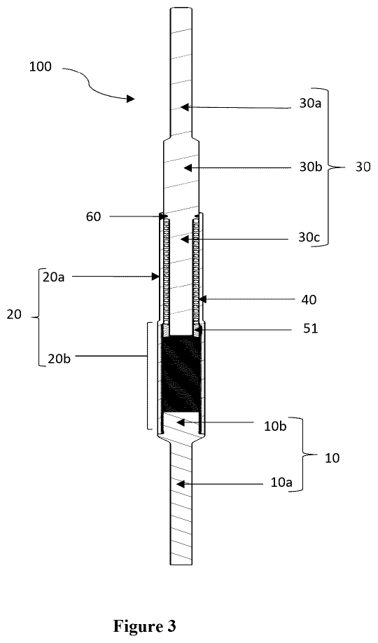

A first embodiment of the present system (100), as illustrated in FIGS. 2 and 3, is described herein below. In accordance with the first embodiment, the present disclosure provides a constant distraction force driven self-actuating growing rod system (100) comprising at least one static rod component (10); at least one hollow intermediate rod component (20); at least one growth rod component (30); at least one compression spring component (40); at least one magnetic field-based spring actuation mechanism (50) and at least one dynamic sealing plug (60).

The static rod component (10) of the first embodiment comprises a first static segment (10a) and a second static segment (10b). The first static segment (10a) is adapted to be affixed to at least one bony anatomy at a first pre-determined location, which is below the deformity site. The second static segment (10b) is threaded along the external circumference and is adapted to couple threadedly with the intermediate rod component (20). The threaded coupling is supplemented by welding.

The hollow intermediate rod component (20) of the first embodiment comprises a first intermediate segment (20a) and a second intermediate segment (20b). The second intermediate segment (20b) is threaded along the internal circumference and is adapted to regulate the upward movement of the magnetic spring actuator component (51) and also facilitate threaded coupling with the second static segment (10b) of the static rod component (10). The hollow intermediate rod component (20) is affixed to the static rod component (10) by threaded coupling and welding such that after such affixation, the intermediate rod component (20) can't rotate with respect to the static rod component (10). In one embodiment, the hollow intermediate rod component (20) is adapted to house the compression spring component (40), the magnetic spring actuator component (51) and the growth rod component (30).

The growth rod component (30) of the first embodiment comprises a first growth segment (30a), a second growth segment (30b) and a third growth segment (30c). The first growth segment (30a) is adapted to be affixed to at least one bony anatomy at a second pre-determined location, which is above the deformity site. The second (30b) and third (30c) growth segments are disposed within the intermediate rod component (20) and configured to telescope out thereof pursuant to the natural growth of the bony anatomy. The growth rod component (30) is connected to the intermediate rod component (20) through a cylindrical joint which allows the motion of the growth rod component (30) with respect to the intermediate rod component (20).

The compression spring component (40) of the first embodiment comprises one compression spring (40) that is coaxially disposed within the intermediate rod component (20), around the third growth segment (30c) of the growth rod component (30) in a pre-compressed state (illustrated in FIG. 2) and sandwiched between the second growth segment (30b) of the growth rod component (30) on a first side and the magnetic spring actuator component (51) on a second side. Since the compression spring component (40) is installed in the system (100) in a pre-compressed state, it is adapted to apply an active distraction force onto the system (100) of the present disclosure, right from the time when the surgeon manually corrects the deformity, followed by affixing the present system (100) onto the corrected bony anatomy. Typically, the compression spring component (40) is at least one selected from the group consisting of a helical coil spring, a conical coil spring, a leaf spring, a constant force spring, a disc spring, a constant rate spring, an extension coil spring and a constant torque spring.

The magnetic field-based spring actuation mechanism (50) of the first embodiment comprises at least one magnetic spring actuator component (51) and at least one magnetic field generating component (not shown in the figures).

The magnetic spring actuator component (51) is threaded along the external circumference and is disposed within the hollow portion of the intermediate rod component (20) in the second intermediate segment (20b), such that one end of the compression spring component (40) rests thereon. The magnetic spring actuator component (51) is adapted to move in at least one motion selected from the group consisting of rotational motion and translational motion. Further, the direction of rotation of the magnetic spring actuator component (51) determines whether the compression spring component (40) gets compressed or relaxed. In one embodiment, the magnetic spring actuator component (51) is adapted to compress the compression spring component (40) upon rotating in a clockwise direction. In another embodiment, the magnetic spring actuator component (51) is adapted to release or cause the compression spring component (40) to relax upon rotating in an anti-clockwise direction.

The magnetic field generating component is located outside the body. The magnetic field generating component is adapted to influence the movement of the magnetic spring actuator component (51) when placed on the back of the patient in contact with skin where the present system (100) is affixed; which in turn compresses the compression spring (41); thereby reinstating the distraction force.

The dynamic sealing plug (60) is affixed between the inner circumference of the first intermediate segment (20a) of the intermediate rod component (20) and the outer circumference of the second growth segment (30b) of the growth rod component (30) and is adapted to prevent movement of at least one contaminant in and out of the system (100). The dynamic sealing plug (60) is at least one selected from the group consisting of O-ring(s), lip seal(s), quad seal(s) or any suitable seals that suit the present application.

During the natural growth of the bony anatomy, the growth rod component (30) telescopes out of the intermediate rod component (20), causing the pre-compressed compression spring (40) to relax; thereby creating a distraction force deficit in the system (100). The distraction force deficit is corrected by causing the relaxed compression spring (40) to get regain compression (illustrated in FIG. 3) by means of the magnetic spring actuator component (51), under the stimulus of magnetic field generating component. The magnetic field generating component causes the magnetic spring actuator component (51) to rotate upwards along the longitudinal axis and along the internal threading of the hollow second intermediate segment (20b) thereby compressing the spring (40) and reinstating an active distraction force onto the system (100).

In post-operative follow ups, a surgeon will take an x-ray of the affected bony anatomy to determine how much growth has occurred. If the surgeon feels the need to increase the constant distraction force, he will perform a non-invasive procedure, by placing the magnetic field generating component on the back of the patient in contact with skin where the system (100) has been affixed. The magnetic field generating component moves the magnetic spring actuator component (51) that compresses the spring (41); thereby increasing and reinstating the distraction force.

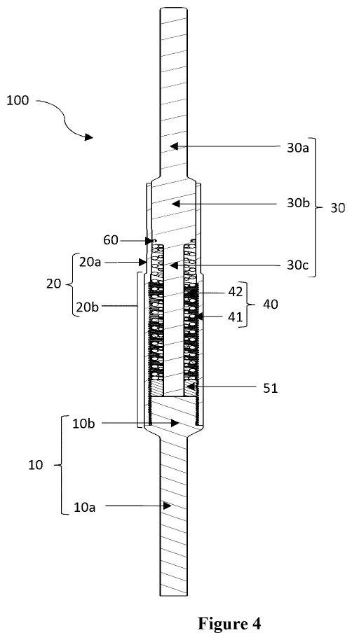

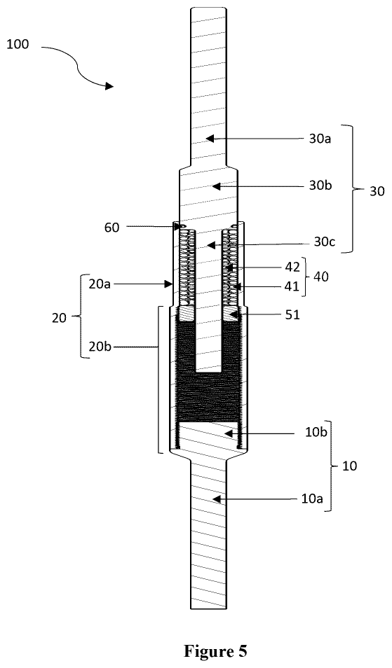

In a second embodiment, as illustrated in FIGS. 4 and 5, the present system (100) comprises the same components, construction, and functioning, except for the compression spring component (40). In a second embodiment, the present system (100) comprises two compression springs (41,42) that are coaxially disposed within the hollow portion of the intermediate rod component (20), around the third growth segment (30c) of the growth rod component (30) and sandwiched between the second growth segment (30b) of the growth rod component (30) on a first side and at least one magnetic spring actuator component (51) at a second side. Typically, the compression spring component (40) is at least one selected from the group consisting of a helical coil spring, a conical coil spring, a leaf spring, a constant force spring, a disc spring, a constant rate spring, an extension coil spring and a constant torque spring.

Typically, the first (41) of the two compression springs has a diameter larger than the diameter of the second (42) of the two compression springs and there is radial clearance therebetween which facilitates the springs (41,42) to work in parallel. At installation, the first (41) of the two compression springs is in a pre-compressed state and the second (42) of the two compression springs is in a free state (illustrated in FIG. 4). At installation the second spring (42) is free, because the required force is provided by the first spring (41) singularly. During implantation of the system (100) disclosed in the second embodiment, the first spring (41) is pre-compressed to provide initial distraction force. After certain growth of the spine, the growing rod component (30) extends from its installation position, causing the pre-compressed spring to relax, which leads to reduction in the distraction force. Hence to re-instate the distraction force, the first compression spring (41) is made to regain compression. This is achieved by rotating the magnetic spring actuator component (51) in the clock-wise direction. To cater to the requirement of maintaining additional distraction force, consequent to additional growth of the spine, which the first spring (41) alone can't balance out, the second spring (42) is included in the system (100). The purpose of inclusion of the second spring (42) in addition to the first (41), is to provide further additional force within the same sized cylinder which is the growing rod system (100), wherein the second compression spring (41) is also compressed by rotating the magnetic spring actuator component (51) in the clock-wise direction (illustrated in FIG. 5).

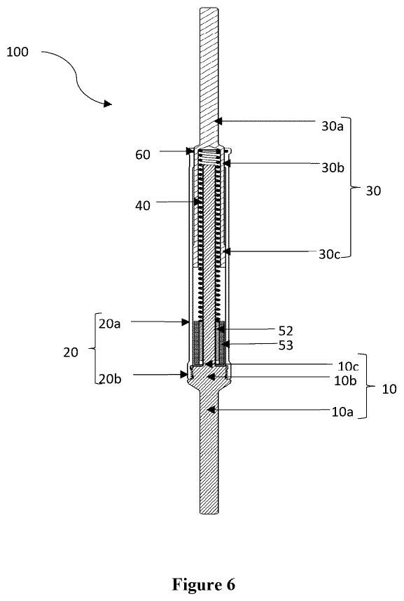

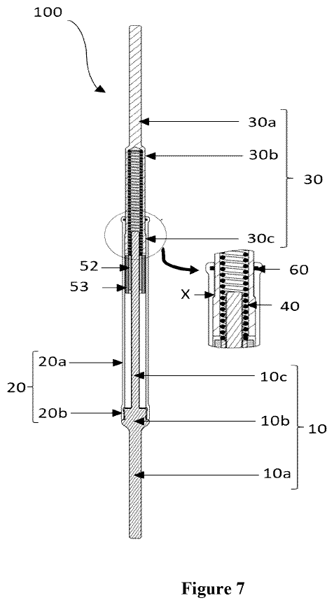

A third embodiment of the system (100), as illustrated in FIGS. 6 and 7 comprises at least one static rod component (10), at least one hollow intermediate rod component (20), at least one growth rod component (30), at least one compression spring component (40), at least one magnetic field-based spring actuation mechanism (50) and at least one dynamic sealing plug (60).

The static rod component (10) of the third embodiment is partially threaded along the external circumference and comprises a first static segment (10a) and a second static segment (10b) and a third static segment (10c). The first static segment (10a) is adapted to be affixed to at least one bony anatomy at a first pre-determined location, which is below the deformity site. The second static segment (10b) is threaded along the external circumference and is adapted to couple threadedly with the intermediate rod component (20), followed by welding, which ensures that the intermediate rod component (20) doesn't rotate with respect to the static rod component (10). The third static segment (10c) is threaded along the external circumference and is adapted to regulate the upward movement of the spring actuator component (52).

The hollow intermediate rod component (20) of the third embodiment is threadedly coupled with the static rod component (10) and adapted to house the compression spring component (40), the spring actuator component (52), at least one magnetic component (53) and the growth rod component (30). The hollow intermediate rod component (20) comprises a first intermediate segment (20a) and a second intermediate segment (20b). The second intermediate segment (20b) is threaded along the internal circumference and is adapted to threadedly couple with the second static segment (10b) of the static rod component (10), followed by welding.

The growth rod component (30) of the third embodiment, is disposed within the intermediate rod component (20) and is configured to envelope the compression spring component (40) and telescope out of the intermediate rod component (20) pursuant to the natural growth of the bony anatomy. The growth rod component (30) comprises a first growth segment (30a) and a second growth segment (30b) and a third growth segment (30c). The first growth segment (30a) is adapted to be affixed to at least one bony anatomy at a second pre-determined location, which is above the deformity site. The second (30b) and third growth segment (30c) are disposed within the intermediate rod component (20), are hollow and envelope the compression spring component (40) and are configured to telescope out of the intermediate rod component (20), pursuant to the natural growth of the bony anatomy. The growth rod component (30) is connected to the intermediate rod component (20) through a cylindrical joint which allows the motion of the growth rod component (30) with respect to the intermediate rod component (20).

Characteristically, a part of the internal circumference of the first segment (20a) of the intermediate rod component (20) and the external circumference of the third segment (30c) of the growth rod component (30) have complementary profiles, that fit perfectly into each other, functioning as a physical stop (X) adapted to limit the growth rod (30) from telescoping completely out of the intermediate rod (20).

The compression spring component (40) of the third embodiment, comprises one compression spring (41) that is coaxially disposed within the intermediate rod component (20), further within the second growth segment (30b) of the growth rod component (30); thereby sandwiched between the second growth segment (30b) of the growth rod component (30) on a first side and the spring actuator component (52) on a second side, in a pre-compressed state (illustrated in FIG. 6). Since the compression spring component (40) is installed in the system (100) in a pre-compressed state, it is adapted to apply an active distraction force onto the system (100) of the present disclosure, right from the time when the surgeon manually corrects the deformity, followed by affixing the present system (100) onto the corrected bony anatomy. Typically, the compression spring component (40) is at least one selected from the group consisting of a helical coil spring, a conical coil spring, a leaf spring, a constant force spring, a disc spring, a constant rate spring, an extension coil spring and a constant torque spring.

The magnetic field-based spring actuation mechanism (50) of the third embodiment comprises at least one spring actuator component (52), at least one magnetic component (53) and at least one magnetic field generating component (not shown in the figures).

The spring actuator component (52) is disposed within the hollow portion of the intermediate rod component (20) in the first intermediate segment (20a), such that one end of the compression spring component (40) rests on the spring actuator component (52). The spring actuator component (52) has an internal bore and is threaded along the internal circumference and is adapted to move upwards along the longitudinal axis and along the external threading of the static rod component (10), consequent to the movement of the magnetic component (53), under the stimulus of the magnetic field generating component, which compresses the compression spring component (40). The spring actuator component (52) being adapted to move in at least one motion selected from the group consisting of rotational motion and translational motion.

The magnetic component (53) is disposed within the hollow portion of the intermediate rod component (20) in the first intermediate segment (20a) and positioned such that there is an air gap between magnetic component (53) and the and static rod component (10). The magnetic component (53) has an internal bore and is press-fitted with the spring actuator component (52) along the external circumference and is adapted to move in at least one motion selected from the group consisting of rotational motion and translational motion.

Further, the direction of rotation of the magnetic component (53) determines whether the compression spring component (40) gets compressed or extended. In one embodiment, the magnetic component (53) and consequently, the spring actuator component (52) are adapted to compress the compression spring component (40) upon rotating in a clockwise direction. In another embodiment, the magnetic component (53) and consequently, the spring actuator component (52) are adapted to release or cause the compression spring component (40) to relax, upon rotating in an anti-clockwise direction.

The magnetic field generating component is located outside the body. The magnetic field generating component is adapted to influence the movement of the magnetic component (53) and consequently the spring actuator component (52), when placed on the back of the patient in contact with skin where the present system (100) is affixed; which in turn compresses the compression spring (41); thereby reinstating the distraction force.

The dynamic seal (60) is affixed between the inner circumference of the first intermediate segment (20a) of the intermediate rod component (20) and the outer circumference of the second growth segment (30b) of the growth rod component (30) and is adapted to prevent movement of at least one contaminant in and out of the system (100). The dynamic sealing plug (60) is at least one selected from the group consisting of O-ring(s), lip seal(s), quad seal(s) or any suitable seals that suit the present application.

During the natural growth of the bony anatomy, the growth rod component (30) telescopes out of the intermediate rod component (20), causing the pre-compressed compression spring (40) to relax; thereby creating a distraction force deficit in the system (100). The distraction force deficit is corrected by manipulating the magnetic component (53) to cause the spring actuator component (52) to move upwards along the longitudinal axis and along the external threading of the third segment of the static rod component (10c), under the stimulus of the magnetic field generating component; thereby causing the relaxed compression spring component (40) to regain compression (as illustrated in FIG. 7) and reinstate and maintain an active distraction force onto the system (100).

In post-operative follow ups, a surgeon will take an x-ray of the affected bony anatomy to determine how much growth has occurred. If the surgeon feels the need to increase the constant distraction force, he will perform a non-invasive procedure, by placing the magnetic field generating component on the back of the patient in contact with skin where the system (100) has been affixed. The magnetic field generating component causes the magnetic component (53) and consequently, the spring actuator component (52) to compress the compression spring component (40); thereby increasing and reinstating the distraction force.

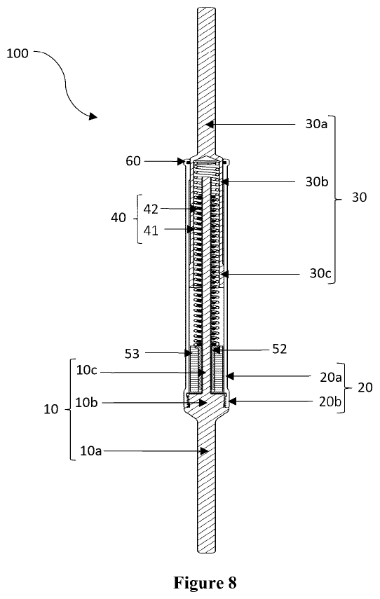

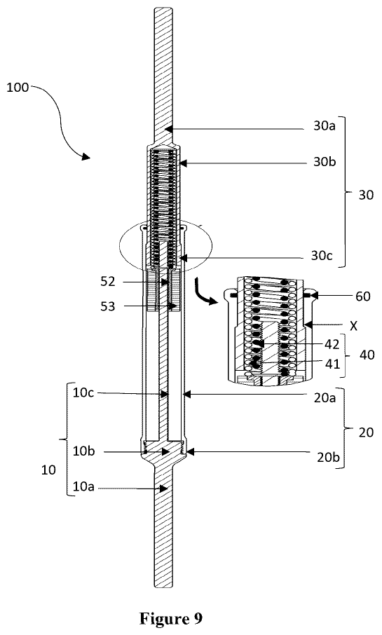

In a fourth embodiment, illustrated in FIGS. 8 and 9, the present system (100) comprises the same components, construction and functioning as that of the third embodiment, except for the compression spring component (40). In the fourth embodiment, the present system (100) comprises two compression springs (41,42) that are coaxially disposed within the hollow portion of the intermediate rod component (20), further within the second growth segment (30b) of the growth rod component (30) and sandwiched between the second growth segment (30b) of the growth rod component (30) on a first side and the spring actuator component (52) and the magnetic component (53) at a second side. Typically, the compression spring component (40) is at least one selected from the group consisting of a helical coil spring, a conical coil spring, a leaf spring, a constant force spring, a disc spring, a constant rate spring, an extension coil spring and a constant torque spring.

Typically, the first (41) of the two compression springs (41,42) has a diameter larger than the diameter of the second (42) of the two compression springs (41,42) and there is radial clearance therebetween which facilitates the springs to work in parallel. At installation, the first (41) of the two compression springs is in a pre-compressed state and the second (42) of the two compression springs is in a free state (as illustrated in FIG. 8). At installation the second spring (42) is free, because the required force is provided by the first spring (41) singularly. During implantation of the system (100) disclosed in the fourth embodiment, the first spring (41) is pre-compressed to provide initial distraction force. After certain growth of the spine, the growing rod component (30) extends from its installation position, causing the pre-compressed spring to relax, which leads to reduction in the distraction force. Hence to re-instate the distraction force, the first compression spring (41) is made to regain compression. This is achieved by rotating the magnetic component (53) and the spring actuator component (52) in the clock-wise direction. To cater to the requirement of maintaining additional distraction force, consequent to additional growth of the spine, which the first spring (41) alone can't balance out, the second spring (42) is introduced. The purpose of inclusion of the second spring (42) in addition to the first (41), is to provide further additional force within the same sized cylinder which is the growing rod system (100), wherein the second compression spring (42) is also compressed by rotating the magnetic component (53) and the spring actuator component (52) in the clock-wise direction (illustrated in FIG. 9)

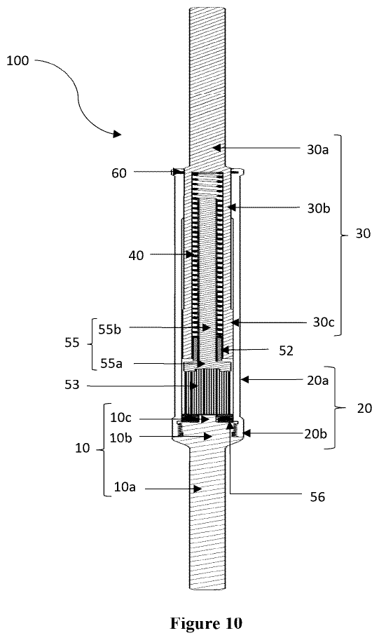

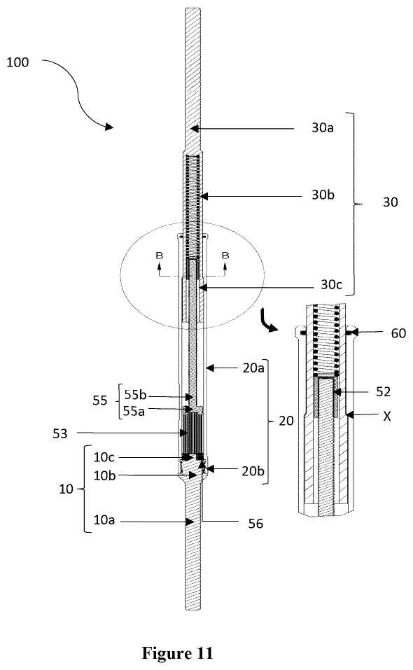

A fifth embodiment of the present system (100), as illustrated in FIGS. 10 and 11, is described herein below. The fifth embodiment of the present system (100) comprises at least one static rod component (10); at least one hollow intermediate rod component (20), at least one growth rod component (30); at least one compression spring component (40); at least one magnetic field-based spring actuation mechanism (50) and at least one dynamic sealing plug (60). The magnetic field-based spring actuation mechanism (50) of the fifth embodiment comprises at least one spring actuator component (52), at least one magnetic component (53), at least one actuator-magnet connector (55), at least one thrust bearing (56) and at least one magnetic field generating component.

The static rod component (10) of the fifth embodiment comprises a first static segment (10a) and a second static segment (10b) and a third static segment (10c). The first static segment (10a) is adapted to be affixed to at least one bony anatomy at a first pre-determined location, which is below the deformity site. The second static segment (10b) is threaded along the external circumference and is adapted to couple threadedly with the intermediate rod component (20) followed by welding, to ensure that the intermediate rod component (20) can't rotate with respect to the static rod component (10). The third static segment (10c) is adapted to be surrounded by at least one thrust bearing (56). The thrust bearing (56) facilitates the smooth rotation of the magnetic component (53).

The intermediate rod component (20) of the fifth embodiment, is threadedly coupled with the static rod component (10) and is adapted to house the compression spring component (40), the spring actuator component (52), the magnetic component (53), the actuator-magnet connector (55) and the growth rod component (30). The hollow intermediate rod component (20) of the fifth embodiment comprises a first intermediate segment (20a) and a second intermediate segment (20b). The second intermediate segment (20b) is threaded along the internal circumference is adapted to threadedly couple with the second static segment (10b) of the static rod component (10).

The growth rod component (30) of the fifth embodiment, is disposed within the intermediate rod component (20) and is configured to envelope the compression spring component (40) and telescope out of the intermediate rod component (20) pursuant to the natural growth of the bony anatomy. The growth rod component (30) comprises a first growth segment (30a), a second growth segment (30b) and a third growth segment (30c). The first growth segment (30a) is adapted to be affixed to at least one bony anatomy at a second pre-determined location, which is above the deformity site. The second growth segment (30b) is disposed within the intermediate rod component (20), is hollow and envelope the compression spring component (40). Furthermore, the growth rod component has at least one flat profiles (Y) along the internal circumference and is configured to telescope out of the intermediate rod component (20) pursuant to the natural growth of the bony anatomy. The growth rod component (30) is connected to the intermediate rod component (20) through a cylindrical joint which allows the motion of the growth rod component (30) with respect to the intermediate rod component (20).

Characteristically, a part of the internal circumference of the first segment (20a) of the intermediate rod component (20) and the external circumference of the third segment (30c) of the growth rod component (30) have complementary profiles, that fit perfectly into each other, functioning as a physical stop (X) adapted to limit the growth rod (30) from telescoping completely out of the intermediate rod (20).

The compression spring component (40) of the fifth embodiment, comprises one compression spring (40) that is coaxially disposed within the intermediate rod component (20), further within the second growth segment (30b) of the growth rod component (30) and around the actuator-magnet connector (55); thereby sandwiched between the second growth segment (30b) of the growth rod component (30) on a first side and the spring actuator component (52) on a second side, in a pre-compressed state (as illustrated in FIG. 10). Since the compression spring component (40) is installed in the system (100) in a pre-compressed state, it is adapted to apply an active distraction force onto the system (100) of the present disclosure, right from the time when the surgeon manually corrects the deformity, followed by affixing the present system (100) onto the corrected bony anatomy. Typically, the compression spring component (40) is at least one selected from the group consisting of a helical coil spring, a conical coil spring, a leaf spring, a constant force spring, a disc spring, a constant rate spring, an extension coil spring and a constant torque spring.

In the magnetic field-based spring actuation mechanism (50) of the fifth embodiment comprises a spring actuator component (52), a magnetic component (53) and a actuator-magnet connector (55), that are disposed within the hollow portion of the intermediate rod component (20), such that one end of the compression spring component (40) rests on the spring actuator component (52). the magnetic field-based spring actuation mechanism (50) of the present embodiment also comprises a magnetic field generating component (not shown in the figures).

The spring actuator component (52) is disposed within the hollow portion of the intermediate rod component (20) in the first intermediate segment (20a). The spring actuator component (52) has an internal bore and at least one flat profile (Z) along the external circumference and is threaded along the internal circumference. The spring actuator component (52) is adapted to translate upwards along the longitudinal axis and along the external threading of the actuator-magnet connector (55), consequent to the movement of the magnetic component (53), under the stimulus of the magnetic field generating component and compress the relaxed part of compression spring component (40), during natural growth.

Typically, the flat profile (Y) along the internal circumference of the second (30b) and third growth segment (30c) of the growth rod component (30) and the flat profile (Z) along the external circumference of the spring actuator complement (52) are complementary.

The magnetic component (53) is solid and cylindrical and rests on the thrust bearing (56) on one side and is in connection with the actuator-magnetic connector (55) on the other side and is adapted to move in a rotational motion.

The actuator-magnet connector (55) comprises a first actuator-magnet connector segment (55a) and a second actuator-magnet connector segment (55b); wherein the first actuator-magnet connector segment (55a) is in direct contact with the magnetic component (53) and the second actuator-magnet connector segment (55b) is threaded along the external circumference and is adapted to move in a rotational motion and regulate the upward movement of the spring actuator component (52).

Further, the direction of rotation of the magnetic component (53) determines whether the compression spring component (40) gets compressed or relaxed. In one embodiment, the magnetic component (53) and consequently the actuator-magnet connector (55) and the spring actuator component (52) are adapted to compress the compression spring component (40) upon moving in a clockwise direction. In another embodiment, the magnetic component (53) and consequently, the actuator-magnet connector (55) and the spring actuator component (52) are adapted to release or cause the compression spring component (40) to relax, upon rotating in an anti-clockwise direction.

The magnetic field generating component is located outside the body. The magnetic field generating component is adapted to influence the movement of the magnetic component (53) and consequently the spring actuator component (52) and the actuator-magnet connector (55), when placed on the back of the patient in contact with skin where the present system (100) is affixed; which in turn compresses the compression spring (41); thereby reinstating the distraction force.

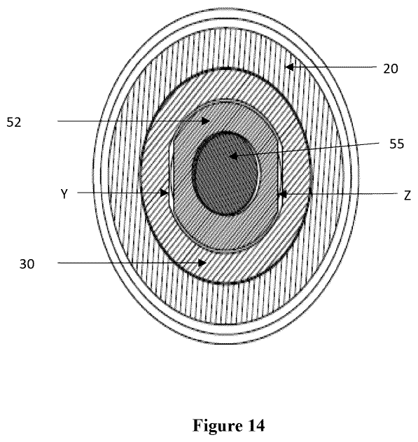

There is a flat profile (Z), as illustrated in FIG. 14, along the external circumference of the spring actuator component (52) which complements the flat profile (Y) along the internal circumference of the second (30b) and third growth segment (30c) of the growth rod component (30). The peculiar design of flat profiles will prohibit the spring actuator component (52) to rotate inside the growth rod component (30) upon magnetic induction, thereby allowing only translational motion of the spring actuator component (52).

The dynamic seal (60) is affixed between the inner circumference of the first intermediate segment (20a) of the intermediate rod component (20) and the outer circumference of the second growth segment (30b) of the growth rod component (30) and is adapted to prevent movement of at least one contaminant in and out of the system (100). The dynamic sealing plug (60) is at least one selected from the group consisting of O-ring(s), lip seal(s), quad seal(s) or any suitable seals that suit the present application.

During the natural growth of the bony anatomy, the growth rod component (30) telescopes out of the intermediate rod component (20), causing the pre-compressed compression spring component (40) to relax; thereby creating a distraction force deficit in the system (100). The distraction force deficit is corrected by manipulating the magnetic component (53) to further manipulate the actuator-magnet connector (55) and cause the spring actuator component (52) to move upwards along the longitudinal axis and along the external threading of the actuator-magnet connector (55), under the stimulus of the magnetic field generating component; thereby causing the relaxed compression spring component (40) to regain compression (as illustrated in FIG. 11) and reinstate the active distraction force onto the system (100).

In post-operative follow ups, a surgeon will take an x-ray of the affected bony anatomy to determine how much growth has occurred. If the surgeon feels the need to increase the constant distraction force, he will perform a non-invasive procedure, by placing the magnetic field generating component on the back of the patient in contact with skin where the system (100) has been affixed. The magnetic field generating component causes the magnetic component (53) and consequently, the spring actuator component (52) to compress the compression spring component (40); thereby increasing and reinstating the distraction force.

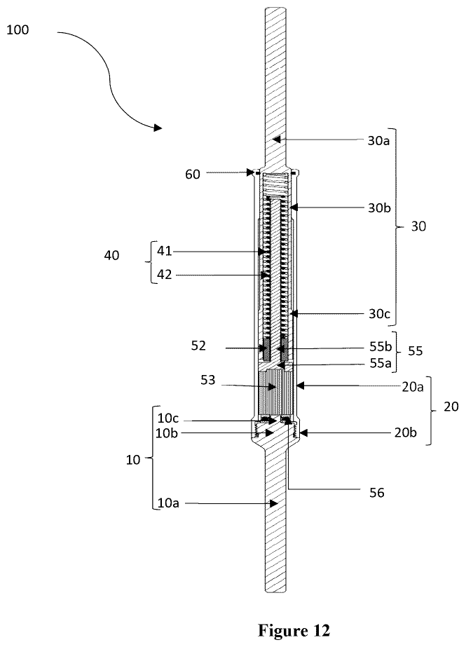

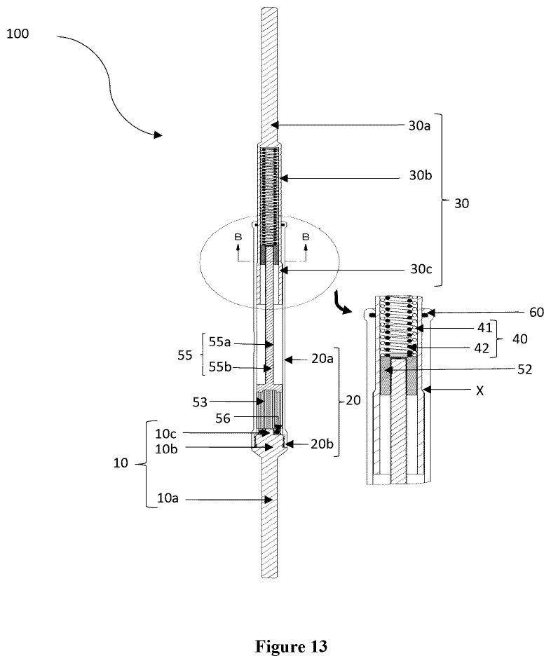

In a sixth embodiment, as illustrated in FIGS. 12 and 13, the present system (100) comprises the same components, construction and functioning as that of the fifth embodiment, except for the compression spring component (40). In a sixth embodiment, the present system (100) comprises two compression springs (41,42) that are installed coaxially within the intermediate rod component (20), further within the second growth segment (30b) of the growth rod component (30) and around the actuator-magnet connector (55); thereby sandwiched between the second growth segment (30b) of the growth rod component (30) on a first side and the spring actuator component (52) on a second side. At installation, the first (41) of the two compression springs (41,42) is in a compressed state and the second (42) of the two compression springs (41,42) is in a free state (as illustrated in FIG. 12). Typically, the first (41) of the two compression springs (41,42) has a diameter larger than the diameter of the second (42) of the two compression springs (41,42) and there is radial clearance therebetween which facilitates the springs to work in parallel. Typically, the compression spring component (40) is at least one selected from the group consisting of a helical coil spring, a conical coil spring, a leaf spring, a constant force spring, a disc spring, a constant rate spring, an extension coil spring and a constant torque spring.

During implantation of the system (100) disclosed in the sixth embodiment, the first spring (41) is pre-compressed to provide initial distraction force. After certain growth of the spine, the growing rod component (30) extends from its installation position, causing the pre-compressed spring to relax, which leads to reduction in the distraction force. Hence to re-instate the distraction force, the first compression spring (41) is made to regain compression. This is achieved by rotating the magnetic component (53) and the actuator-magnet connector (55) in the clock-wise direction, resulting in upward translation of spring actuator component (52). To cater to the requirement of maintaining additional distraction force, consequent to additional growth of the spine, which the first spring (41) alone can't balance out, the second spring (42) is introduced. The purpose of inclusion of the second spring (42) in addition to the first (41), is to provide further additional force within the same sized cylinder which is the growing rod system (100) wherein the second compression spring (42) is compressed by rotating the magnetic component (53) and the actuator-magnet connector (55) in the clock-wise direction (illustrated in FIG. 13).

It is crucial to note that multiple other embodiments of the present system (100) are possible by incorporating a plurality of compression spring components (40).

Furthermore, the magnetic field-based spring actuation mechanism (50) may consist of either one of the magnetic spring actuator component (51) or spring actuator component (52) or magnetic component (53) or actuator-magnet connector (55) or one thrust bearing (56) and magnetic field generating component depending on the embodiment considered.

While embodiments of the present invention have been illustrated and described, various modifications can be made without departing from the scope of the invention. Therefore, the invention should not be limited, except for the following claims and equivalents thereof.

The afore-stated components of the system (100) of the present disclosure are manufactured from biocompatible materials. Further, the components of the system (100) of the present disclosure are manufactured from at least one material selected from the group that includes but is not limited to metal(s), metal alloys, polymers and non-Newtonian fluids. For the purpose of the present disclosure, the term metal is at least one selected from the group that includes but is not limited to titanium, cobalt-chromium-molybdenum, and stainless steel or any other metal or metal alloy suitable from biocompatibility and strength perspective. For the purpose of the present disclosure, the term polymers is at least one selected from the group that includes but is not limited to high density polyethylene (HDPE), polyurethane, polycarbonate urethane, ultra-high molecular weight polyethylene (UHMWPE), polyethylene terephthalate (PET), polyether ether ketone (PEEK) and silicone or any other polymer suitable from biocompatibility and strength perspective. All the components of the system (100) of the present disclosure may be fabricated separately and attached together using conventional manufacturing techniques.

Technical Advantages and Economic Significance

The technical advantages and economic significance of the constant distraction force driven self-actuating growing rod system (100), of the present disclosure are presented herein after:
The present system (100) does not hamper the natural growth of the patient;
The present system (TOO) provides multiple point anchoring to the bony anatomy to reduce the chances of rod breakage;
The present system (100) constantly adapts according to the natural growth of the patient, providing distractions and distraction forces exactly required by the patient's body;
In the present system (100), an external instrument generates magnetic field and interacts with the magnetic component (53) without any physical connection by the phenomenon of magnetic coupling/attraction; thereby minimizing surgical intervention;
The present system (100) causes an improvement in the quality of life of the patient due to non-repetitive surgical distraction;
The present system (100) does not contain any fluids; thereby eliminating the chance of fluid leakage;
The present system (100) light-weight and economical; and
The present system (100) eliminates the risk of tissue necrosis caused due to fluid leakage.

We claim:

1. A constant distraction force driven self-actuating growing rod system (100) for implantation on a deformed bony anatomy; said system (100) comprising:
a. at least one static rod component (10);
b. at least one intermediate rod component (20);
c. at least one growth rod component (30);
d. at least one compression spring component (40) adapted to apply an active distraction force onto the system (100);
e. at least one magnetic field-based spring actuation mechanism (50); and
f. at least one dynamic sealing plug (60),
wherein during the natural growth of the deformed bony anatomy, the growth rod component (30) telescopes out of the intermediate rod component (20), creating a distraction force deficit in the system (100); said distraction force deficit being corrected by causing at least one part of the compression spring component (40) to compress by means of said magnetic field-based spring actuation mechanism (50); thereby maintaining an active distraction force onto the system (100).

2. The system (100) as claimed in claim 1, wherein said compression spring component (40) is at least one selected from the group consisting of a helical coil spring, a conical coil spring, a leaf spring, a constant force spring, a disc spring, a constant rate spring, an extension coil spring and a constant torque spring.

3. The system (100) as claimed in claim 1, wherein a part of said compression spring component (40) is installed in a pre-compressed state and during the natural growth of the deformed bony anatomy, the telescoping movement of the growth rod component (30) causes said pre-compressed part of the compression spring component (40) to relax; thereby creating a distraction force deficit in the system (100).

4. The system (100) as claimed in claim 1, wherein said dynamic sealing plug (60) is affixed between the inner circumference of a first intermediate segment (20a) of the intermediate rod component (20) and the outer circumference of a second growth segment (30b) of the growth rod component (30) and is adapted to prevent movement of at least one contaminant in and out of the system (100).

5. The system (100) as claimed in claim 1, comprising at least one anti-rotation feature selected from the group consisting of gears, splines, keys and ratchet.

6. The system (100) as claimed in claim 1, being adapted for the correction of orthopaedic deformities.

7. A constant distraction force driven self-actuating growing rod system (100) for implantation on a deformed bony anatomy; said system (100) comprising:
a. at least one static rod component (10);
b. at least one hollow intermediate rod component (20), threadedly coupled with the static rod component (10) and adapted to house at least one compression spring component (40), at least one magnetic spring actuator component (51) and at least one growth rod component (30);
c. at least one growth rod component (30) disposed within the intermediate rod component (20) and configured to telescope out thereof pursuant to the natural growth of the bony anatomy;
d. at least one compression spring component (40), being installed coaxially within said intermediate rod component (20) and adapted to apply an active distraction force onto the system (100);
e. at least one magnetic field-based spring actuation mechanism (50) comprising at least one magnetic spring actuator component (51) and at least one magnetic field generating component; wherein said magnetic spring actuator component (51) is disposed within the intermediate rod component (20), such that one end of said compression spring(s) (40) rests thereon; and
f. at least one dynamic sealing plug (60);
wherein during the natural growth of the bony anatomy, the growth rod component (30) telescopes out of the intermediate rod component (20), creating a distraction force deficit in the system (100); said distraction force deficit being corrected by causing at least one part of the compression spring component (40) to get compressed by means of said magnetic spring actuator component (51), under the stimulus of said magnetic field generating component; thereby maintaining an active distraction force onto the system (100).

8. The system (100) as claimed in claim 7, wherein said at least one static rod component (10) comprises a first static segment (10a) and a second static segment (10b), wherein said first static segment (10a) is adapted to be affixed to at least one bony anatomy at a first pre-determined location and said second static segment (10b) is threaded along the external circumference and is adapted to couple threadedly with the intermediate rod component (20).

9. The system (100) as claimed in claim 8, wherein the first pre-determined location of affixation of the first static segment (10a) to the bony anatomy is below the deformity site.

10. The system (100) as claimed in claim 7, wherein said at least one hollow intermediate rod component (20) comprises a first intermediate segment (20a) and a second intermediate segment (20b) wherein said second intermediate segment (20b) is threaded along the internal circumference and is adapted to regulate the upward movement of the magnetic spring actuator component (51) and facilitate threaded coupling with the second static segment (10b) of the static rod component (10), followed by welding.

11. The system (100) as claimed in claim 7, wherein said at least one growth rod component (30) comprises a first growth segment (30a), a second growth segment (30b) and a third growth segment (30c); wherein said first growth segment (30a) is adapted to be affixed to at least one bony anatomy at a second pre-determined location, said second (30b) and third (30c) growth segments are disposed within the intermediate rod component (20) and configured to telescope out thereof pursuant to the natural growth of the bony anatomy.

12. The system (100) as claimed in claim 11, wherein the second pre-determined location of affixation of the first growth segment (30a) to the bony anatomy is above the deformity site.

13. The system (100) as claimed in claim 7, wherein said compression spring component (40) is at least one selected from the group consisting of a helical coil spring, a conical coil spring, a leaf spring, a constant force spring, a disc spring, a constant rate spring, an extension coil spring and a constant torque spring.

14. The system (100) as claimed in claim 7, wherein said compression spring component (40) comprises one compression spring (41) that is installed coaxially within said intermediate rod component (20), around the third growth segment (30c) of the growth rod component (30) in a pre-compressed state, and is sandwiched between the second growth segment (30b) of the growth rod component (30) on a first side and the magnetic spring actuator component (51) on a second side.

15. The system (100) as claimed in claim 7, wherein said compression spring component (40) comprises two compression springs (41, 42) being coaxially disposed within said hollow portion of the intermediate rod component (20), around the third growth segment (30c) of the growth rod component (30) and sandwiched between the second growth segment (30b) of the growth rod component (30) on a first side and at least one magnetic spring actuator component (51) at a second side, wherein at installation the first of the two compression springs (41) is in a pre-compressed state and the second of the two compression springs (42) is in a free state.

16. The system (100) as claimed in claim 15, wherein the first of the two compression springs (41) has a diameter larger than the diameter of the second of the two compression springs (42); thereby maintaining radial clearance therebetween.

17. The system (100) as claimed in claim 7, wherein said magnetic spring actuator component (51) is threaded along the external circumference and disposed within said hollow portion of the intermediate rod component (20) in said second intermediate segment (20b) and is adapted to move in at least one motion selected from the group consisting of rotational motion and translational motion.

18. The system (100) as claimed in claim 7, wherein said magnetic spring actuator component (51) is adapted to compress the compression spring component (40) upon rotating in a clockwise direction and said magnetic spring actuator component (51) is adapted to release the compression spring component (40) upon rotating in an anti-clockwise direction.

19. The system (100) as claimed in claim 7, wherein said magnetic field generating component is outside the body.

20. The system (100) as claimed in claim 7, wherein said dynamic sealing plug (60) is affixed between the inner circumference of the first intermediate segment (20a) of the intermediate rod component (20) and the outer circumference of the second growth segment (30b) of the growth rod component (30) and is adapted to prevent movement of at least one contaminant in and out of the system (100).

21. A constant distraction force driven self-actuating growing rod system (100) for implantation on a deformed bony anatomy; said system (100) comprising:
a. at least one static rod component (10), partially threaded along the external circumference;
b. at least one hollow intermediate rod component (20), threadedly coupled with the static rod component (10) and adapted to house at least one compression spring component (40), at least one spring actuator component (52), at least one magnetic component (53) and at least one growth rod component (30);
c. at least one growth rod component (30) disposed within the intermediate rod component (20), configured to envelope the compression spring component (40) and telescope out of the intermediate rod component (20) pursuant to the natural growth of the bony anatomy;
d. at least one compression spring component (40) being disposed coaxially within said intermediate rod component (20) and said growth rod component (30) and adapted to apply an active distraction force onto the system (100);
e. at least one magnetic field-based spring actuation mechanism (50) comprising at least one spring actuator component (52), at least one magnetic component (53) and at least one magnetic field generating component; wherein said spring actuator component (52) and said magnetic component (53) are disposed within the intermediate rod component (20), such that there is an air gap between magnetic component (53) and static rod component (10) and one end of said compression spring component (40) rests on the spring actuator component (52); and
f. at least one dynamic sealing plug (60);
wherein during the natural growth of the bony anatomy, the growth rod component (30) telescopes out of the intermediate rod component (20) creating a distraction force deficit in the system (100); said distraction force deficit being corrected by causing at least one part of the compression spring component (40) to get compressed by means of said spring actuator component (52) and said magnetic component (53), under the stimulus of said magnetic field generating component; thereby maintaining an active distraction force onto the system (100).

22. The system (100) as claimed in claim 21, wherein said at least one static rod component (10) comprises a first static segment (10a), a second static segment (10b) and a third static segment (10c), wherein said first static segment (10a) is adapted to be affixed to at least one bony anatomy at a first pre-determined location, said second static segment (10b) is threaded along the external circumference and is adapted to couple threadedly with the intermediate rod component (20) and said third static segment (10c) is threaded along the external circumference and is adapted to regulate the upward movement of the spring actuator component (52).

23. The system (100) as claimed in claim 22, wherein the first pre-determined location of affixation of the first static segment (10a) to the bony anatomy is below the deformity site.

24. The system (100) as claimed in claim 21, wherein said at least one hollow intermediate rod component (20) comprises a first intermediate segment (20a) and a second intermediate segment (20b) wherein said second intermediate segment (20b) is threaded along the internal circumference and is adapted to threadedly couple with the second static segment (10b) of the static rod component (10), followed by welding.

25. The system (100) as claimed in claim 21, wherein said at least one growth rod component (30) comprises a first growth segment (30a), a second growth segment (30b) and a third growth rod segment (30c); wherein said first growth segment (30a) is adapted to be affixed to at least one bony anatomy at a second pre-determined location, said second (30b) and third growth segment (30c), being disposed within the intermediate rod component (20), are hollow and envelope the compression spring component (40) and are configured to telescope out of the intermediate rod component (20) pursuant to the natural growth of the bony anatomy.

26. The system (100) as claimed in claim 25, wherein the second pre-determined location of affixation of the first growth segment (30a) to the bony anatomy is above the deformity site.

27. The system (100) as claimed in claims 24 and 25, wherein a part of the internal circumference of said first segment (20a) of the intermediate rod component (20) and the external circumference of said third segment (30c) of the growth rod component (30) have complementary profiles functioning as a physical stop (X) adapted to limit the growth rod (30) from telescoping completely out of the intermediate rod component (20).

28. The system (100) as claimed in claim 21, wherein said compression spring component (40) is at least one selected from the group consisting of a helical coil spring, a conical coil spring, a leaf spring, a constant force spring, a disc spring, a constant rate spring, an extension coil spring and a constant torque spring.

29. The system (100) as claimed in claim 21, wherein said compression spring component (40) comprises one compression spring (41) that is installed coaxially within said intermediate rod component (20), further within the second growth segment (30b) of the growth rod component (30), in a pre-compressed state; thereby sandwiched between the second growth segment (30b) of the growth rod component (30) on a first side and the spring actuator component (52) on a second side.

30. The system (100) as claimed in claim 21, wherein said compression spring component (40) comprises two compression springs (41, 42) that are installed coaxially within said intermediate rod component (20), further within the second growth segment (30b) of the growth rod component (30); thereby sandwiched between the second growth segment (30b) of the growth rod component (30) on a first side and the spring actuator component (52) on a second side, wherein at installation the first of the two compression springs (41) is in a pre-compressed state and the second of the two compression springs (42) is in a free state.

31. The system (100) as claimed in claim 30, wherein the first of the two compression springs (41) has a diameter larger than the diameter of the second of the two compression springs (42); thereby maintaining radial clearance therebetween.

32. The system (100) as claimed in claim 21, wherein said spring actuator component (52) is disposed within said hollow portion of the intermediate rod component (20) in said first intermediate segment (20a), has an internal bore and is threaded along the internal circumference and adapted to move upwards along the longitudinal axis and along the external threading of said static rod component (10), consequent to the movement of the magnetic component (53), under the stimulus of said magnetic field generating component and compress the compression spring component (40); said spring actuator component (52) being adapted to move in at least one motion selected from the group consisting of rotational motion and translational motion.

33. The system (100) as claimed in claim 21, wherein said magnetic component (53) is disposed within said hollow portion of the intermediate rod component (20) in said first intermediate segment (20a), has an internal bore and is press-fitted with the spring actuator component (52) along the external circumference and is adapted to move in at least one motion selected from the group consisting of rotational motion and translational motion.

34. The system (100) as claimed in claim 21, wherein said magnetic component (53) and said spring actuator component (52) are cumulatively adapted to compress a part of the compression spring component (40) upon rotating in a clockwise direction and said magnetic component (53) and said spring actuator component (52) are cumulatively adapted to release a part of the compression spring component (40) upon rotating in an anti-clockwise direction.

35. The system (100) as claimed in claim 21, wherein said magnetic field generating component is outside the body.

36. The system (100) as claimed in claim 21, wherein said dynamic sealing plug (60) is affixed between the inner circumference of the first intermediate segment (20a) of the intermediate rod component (20) and the outer circumference of the second growth segment (30b) of the growth rod component (30) and is adapted to prevent movement of at least one contaminant in and out of the system (100).

37. A constant distraction force driven self-actuating growing rod system (100) for implantation on a deformed bony anatomy; said system (100) comprising:
a. at least one static rod component (10);
b. at least one hollow intermediate rod component (20), threadedly coupled with the static rod component (10) and adapted to house at least one compression spring component (40), at least one spring actuator component (52), at least one magnetic component (53), at least one actuator-magnet connector (55) and at least one growth rod component (30);
c. at least one growth rod component (30) disposed within the intermediate rod component (20), configured to envelope the compression spring component (40) and telescope out of the intermediate rod component (20) pursuant to the natural growth of the bony anatomy;
d. at least one compression spring component (40) being disposed coaxially within said intermediate rod component (20) and said growth rod component (30) and adapted to apply an active distraction force onto the system (100);
e. at least one magnetic field-based spring actuation mechanism (50) comprising at least one spring actuator component (52), at least one magnetic component (53), at least one actuator-magnet connector (55), at least one thrust bearing (56) and at least one magnetic field generating component; wherein said spring actuator component (52), said magnetic component (53), said actuator-magnet connector (55) are disposed within the intermediate rod component (20), such that one end of said compression spring component (40) rests on the spring actuator component (52); and
f. at least one dynamic sealing plug (60);
wherein during the natural growth of the bony anatomy, the growth rod component (30) telescopes out of the intermediate rod component (20) creating a distraction force deficit in the system (100); said distraction force deficit being corrected by causing at least one part of compression spring component (40) to get compressed by means of said spring actuator component (52), said actuator-magnet connector (55) and said magnetic component (53), under the stimulus of said magnetic field generating component; thereby maintaining an active distraction force onto the system (100).

38. The system (100) as claimed in claim 37, wherein said at least one static rod component (10) comprises a first static segment (10a), a second static segment (10b) and a third static segment (10c), wherein said first static segment (10a) is adapted to be affixed to at least one bony anatomy at a first pre-determined location, said second static segment (10b) is threaded along the external circumference and is adapted to couple threadedly with the intermediate rod component (20) and said third static segment (10c) is adapted to be surrounded by at least one thrust bearing (56).

39. The system (100) as claimed in claim 38, wherein the first pre-determined location of affixation of the first static segment (10a) to the bony anatomy is below the deformity site.

40. The system (100) as claimed in claim 37, wherein said at least one hollow intermediate rod component (20) comprises a first intermediate segment (20a) and a second intermediate segment (20b) wherein said second intermediate segment (20b) is threaded along the internal circumference and is adapted to threadedly couple with the second static segment (10b) of the static rod component (10), followed by welding.

41. The system (100) as claimed in claim 37, wherein said at least one growth rod component (30) comprises a first growth segment (30a), a second growth segment (30b) and a third growth segment (30c); wherein said first growth segment (30a) is adapted to be affixed to at least one bony anatomy at a second pre-determined location and the said second (30b) and third growth segment (30c) are hollow, envelope the compression spring component (40), have at least one flat profile (Y) along the internal circumference and are disposed within the intermediate rod component (20), configured to telescope out of the intermediate rod component (20) pursuant to the natural growth of the bony anatomy.

42. The system (100) as claimed in claim 41, wherein the second pre-determined location of affixation of the first growth segment (30a) to the bony anatomy is above the deformity site.

43. The system (100) as claimed in claims 40 and 41, wherein a part of the internal circumference of said first segment (20a) of the intermediate rod component (20) and the external circumference of said third segment (30c) of the growth rod component (30) have complementary profiles functioning as a physical stop (X) adapted to limit the growth rod component (30) from telescoping completely out of the intermediate rod component (20).

44. The system (100) as claimed in claim 37, wherein said compression spring component (40) is at least one selected from the group consisting of a helical coil spring, a conical coil spring, a leaf spring, a constant force spring, a disc spring, a constant rate spring, an extension coil spring and a constant torque spring.

45. The system (100) as claimed in claim 37, wherein said compression spring component (40) comprises one compression spring (41) that is installed coaxially within said intermediate rod component (20), further within the second growth segment (30b) of the growth rod component (30) and around the actuator-magnet connector (55) in a pre-compressed state; thereby sandwiched between the second growth segment (30b) of the growth rod component (30) on a first side and the spring actuator component (52) on a second side.

46. The system (100) as claimed in claim 37, wherein said compression spring component (40) comprises two compression springs (41, 42) that are installed coaxially within said intermediate rod component (20), further within the second growth segment (30b) of the growth rod component (30) and around the actuator-magnet connector (55); thereby sandwiched between the second growth segment (30b) of the growth rod component (30) on a first side and the spring actuator component (52) on a second side, wherein at installation the first of the two compression springs (41) is in a pre-compressed state and the second of the two compression springs (42) is in a free state.

47. The system (100) as claimed in claim 46, wherein the first of the two compression springs (41) has a diameter larger than the diameter of the second of the two compression springs (42); thereby maintaining radial clearance therebetween.

48. The system (100) as claimed in claim 37, wherein said spring actuator component (52) is disposed within said hollow portion of the intermediate rod component (20) in said first intermediate segment (20a), has an internal bore and at least one flat profile (Z) along the external circumference, is threaded along the internal circumference and adapted to translate upwards along the longitudinal axis and along the external threading of said actuator-magnet connector (55), consequent to the movement of the magnetic component (53) under the stimulus of said magnetic field generating component and compress the extended part of compression spring component (40).

49. The system (100) as claimed in claims 41 and 48, wherein the flat profile (Y) along the internal circumference of said second (30b) and third growth segment (30c) of the growth rod component (30) and the flat profile (Z) along the external circumference of the spring actuator component (52) are complementary.

50. The system (100) as claimed in claim 37, wherein said magnetic component (53) is solid and cylindrical and rests on said thrust bearing (56) on one side and is in connection with the actuator-magnetic connector (55) on the other side and is adapted to move in a rotational motion.

51. The system (100) as claimed in claim 37, wherein said actuator-magnet connector (55) comprises a first actuator-magnet connector segment (55a) and a second actuator-magnet connector segment (55b); wherein said first actuator-magnet connector segment (55a) is in direct contact with the magnetic component (53) and said second actuator-magnet connector segment (55b) is threaded along the external circumference and is adapted to move in a rotational motion and regulate the upward movement of the spring actuator component (52).

52. The system (100) as claimed in claim 37 wherein said magnetic component (53), said spring actuator component (52) and said actuator-magnet connector (55) are cumulatively adapted to compress a part of compression spring component (40) upon moving in a clockwise direction and said magnetic component (53), said spring actuator component (52) and said actuator-magnet connector (55) are cumulatively adapted to release a part of compression spring component (40) upon moving in an anti-clockwise direction.

53. The system (100) as claimed in claim 37, wherein said thrust bearing (56) facilitates the smooth rotation of the magnetic component (53).

54. The system (100) as claimed in claim 37, wherein said magnetic field generating component is outside the body.

55. The system (100) as claimed in claim 37, wherein said dynamic sealing plug (60) is affixed between the inner circumference of the first intermediate segment (20a) of the intermediate rod component (20) and the outer circumference of the second growth segment (30b) of the growth rod component (30) and is adapted to prevent movement of at least one contaminant in and out of the system (100).
